(19) United States
(12) Patent Application Publication
(10) Pub. No.: US 2019/0339316 A1
(43) Pub. Date: Nov. 7, 2019

(54) DEVICE FOR MEASURING RADIOELECTRIC DOSE ON A SURFACE, IN PARTICULAR FOR MEASURING THE RADIOLOGICAL DOSE OF A PATIENT DURING A RADIOLOGY OPERATION

(71) Applicants: BIOMEDIQA, Villeneuve D'Ascq (FR); CENTRE NATIONAL DE LA RECHERCHE SCIENTIFIQUE, Paris Cedex 16 (FR)

(72) Inventors: Fouad MAALOUL, Marcq En Baroeul (FR); Laura GUERIN, Lille (FR); Christophe LOYEZ, Festubert (FR)

(21) Appl. No.: 16/467,553

(22) PCT Filed: Dec. 8, 2017
(86) PCT No.: PCT/IB2017/057750

§ 371 Date: Jun. 7, 2019

(30) Foreign Application Priority Data
Dec. 9, 2016 (FR) 16/70747

(51) Int. Cl. G01R 29/08; G01R 15/26; A61B 6/00

(52) U.S. Cl. CPC G01R 29/0857; A61B 6/56; A61B 6/4266; G01R 15/26

(57) ABSTRACT

A device for measuring a radioelectric dose on a surface, in particular for measuring the radiological dose for a patient during a radiology operation comprising a matrix with sensors equipped with transparent radio antennas, capable of creating an electric signal corresponding to the electromagnetic radiation received, means for measuring the electric flows emitted by each sensor and means for processing the flow measurements for calculating the accumulation of doses received by predetermined zones of the matrix.

The present invention relates to a device for measuring a radio dose on a surface, in particular for measuring the radiological dose of a patient during a radiological operation.

### TECHNICAL AREA

The invention lies in the technical field of measuring the radiological doses received by a patient during one or more operations, the operation may be a simple radiology or computerized tomography (CT) scan or a radiological treatment, external or internal or an interventional radiology operation.

This application is however not limited, and the measuring device can be used for the measurement of radiological doses in other applications and in particular for the medical personnel of the radiology departments or generally for the personnel working in sectors with radio risks and in particular in industry.

### PRIOR ART

The measurement of the radio dose received by a patient is a very important medical data especially as medical applications of electromagnetic or ionizing radiation are more numerous in terms of applications in the field of examination and treatment or the help during a surgical procedure.

To date, one of the methods used to evaluate the dose received by a patient is to quantify the average dosimetry for a given radiology or CT scan and to cumulate the estimated doses for each operation or act performed on the patient. This approximate method does not allow a precise estimate of the dose received by the patient, nor does it make it possible to differentiate the doses received by the various parts of the patient's body.

Another method is to estimate the dose received by the patient according to the dose of radiation emitted by the device. Here again the method is imprecise, on the one hand the estimate of the emitted radiation is difficult given the lack of normalization of the calculation for the different types of ionizing apparatus and, on the other hand, the radiation emitted by these devices do not accurately reflect the doses received by the patient.

Indeed, the doses of ionizing radiation are not all received by the patient, in particular considering the effects of dispersion, the position of the body and the distance relative to the device, finally the duration of exposure is not measurable precisely. In addition to this inaccuracy, it is also not possible, like the first method, to precisely identify exposed or overexposed body parts in relation to other parts and in particular at the junction zones, between different incidences.

The consequence of this lack of precise monitoring of the doses received by the patients is on the one hand the risk of exceeding the doses recommended for the patient, and in particular on areas irradiated several times and on the other hand the risk of not practicing, a necessary examination to avoid exceeding the recommended doses even when the patient has received much lower doses.

A device for accurate measurement of this cumulative dose in real time would provide a double benefit for the patient, it would reduce the risk of occurrence of side effects, guiding the doctor in real time in the choice of incidences of radiation beams depending on the maximum doses delivered to the skin of the patient, it would also achieve the diagnostic or therapeutic goal sought by the doctor by assessing the risk of overexposure of the patient to its true value.

### OBJECT OF THE INVENTION

A first object of the present invention is to solve all, or part of the technical problems related to the aforementioned prior art. Another object of the present invention is to provide a device for accurately measuring the dose received by a patient during an operation performed on a patient,

Another object of the present invention is to propose a device making it possible to measure the dose received by a patient that does not modify the operation of ionizing devices and that has no impact on the quality of the images or sequences obtained by the imaging devices.

Another object of the present invention is to provide a device for directly determining the radiation levels received by different areas of the body of the patient.

Another object of the present invention is to provide a device for reliable measurement while being both simple to use and installation on the patient.

### SUMMARY OF THE INVENTION

The present invention relates to a device for measuring a radio dose on a surface, in particular for measuring the radiological dose of a patient during a radiology operation comprising, according to the invention, a matrix with sensors equipped with transparent radio antennas able to create an electrical signal corresponding to the electromagnetic radiation received, means for measuring the electrical fluxes emitted by each sensor and processing means of flow measurement for calculating the accumulation of doses received by predetermined areas of the matrix.

### Definitions

The term “matrix” defines, within the meaning of the present invention, a support for the sensors making it possible to keep the latter together in a set organized regularly or not, or homogeneously or not.

### BRIEF DESCRIPTION OF THE FIGURES

The present invention will be better understood on reading a detailed example of embodiment with reference to the appended figures, provided by way of non-limiting example, among which:

FIG. 1 represents a schematic exemplary embodiment of a device according to the invention,

FIG. 2 represents, schematically and in perspective, a device according to the invention arranged on a patient during a radiology operation,

FIG. 3 represents a photograph of an exemplary embodiment of a radio sensor,

FIG. 4 represents in schematic form an embodiment of the radio sensor.

### DETAILED DESCRIPTION OF THE INVENTION

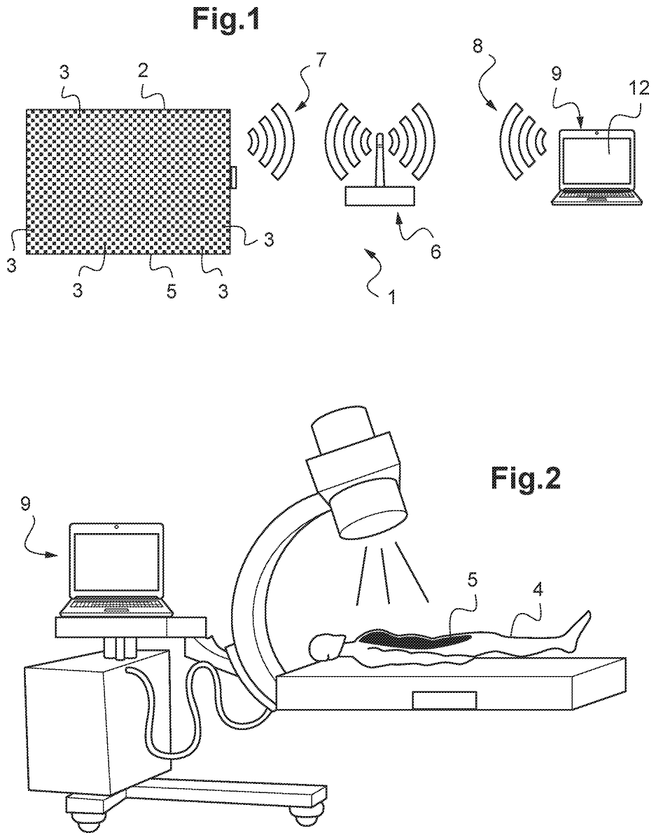

Referring to FIG. 1, there is shown a measuring device 1, the measuring device comprises a matrix 2 with sensors 3, each equipped with a radio-transparent antenna. The number of sensors 3 depends in particular on the precision required for the measurement of the radiations, advantageously the sensors 3 are distributed on the surface of the matrix 2 according to a density of the order of one sensor per cm².

These sensors 3 are able to create an electrical signal corresponding to the received electromagnetic radiation, in this way it is possible to accurately measure the radiation absorbed by the sensor and the radiation transmitted to the body of the patient in contact with the matrix.

The size and shape of the matrix 2 are variable and depend on the part of the body of the patient to be covered. For example, referring to FIG. 2, we see a matrix 2 whose dimensions make it possible to measure radiations substantially at the level of the trunk of a patient 4.

Other forms of matrix 2 will be more suitable for covering the lower limbs or the entire body of the patient.

Advantageously, the matrix 2 is integrated in a mat 5, flexible and transparent radio. The mat 5 is able to conform to the shapes of a patient. The advantage of integrating the matrix 2 in a mat 5 is to be able to clean or wash the latter without the risk of damaging the sensors 3.

In an advantageous variant, the mat 5 comprises locating means, (not shown in the attached figures) able to allow positioning on the patient. For example, the mat 5 may indicate a positioning point relative to the patient's anatomy, including the sternum for the trunk. In this way, it is possible to position the mat 5 incorporating the matrix 2 in the same way during several operations.

Other means of identification are also conceivable and for example means using straps allowing both precise placement and maintenance of the mat 5 on the patient 4.

Referring to FIG. 1, there is shown means 6 for measuring the electrical flux of each sensor 3. These measurement means 6 comprise in particular a multi-input flowmeter, they are associated with first transmission means 7 for the transmission of the streams coming from the sensors 3 and the second transmission means 8 to the processing means 9 of the flow measurements. These processing means 9 allow to calculate the accumulation of the doses received by predetermined zones of the matrix 2.

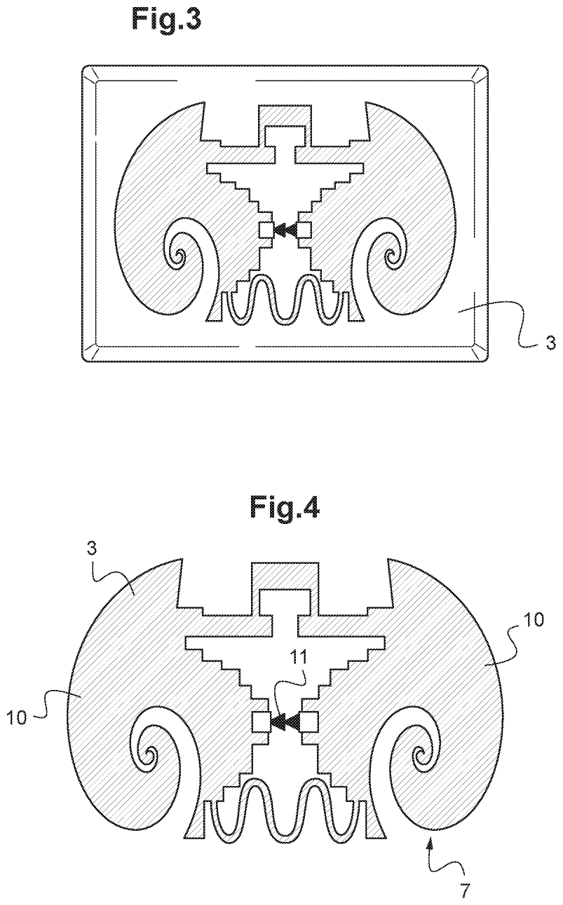

Referring now to FIG. 3, an exemplary embodiment of a sensor 3 with the first transmission means 7 is shown in the form of a photograph.

This sensor 3 allows to generate a signal corresponding to the radiation received, the sensor 3 comprises an antenna 10 associated with a transmitter 11 of RFID type (radio frequency identification device) constituting the first transmission means 7.

Preferably this antenna 10 associated with the transmitter 11 is optimized to operate in ISM bands (industrial, scientific and medical), broadband: Ultra-High Frequencies (860-960 MHz).

The antenna 10 is made of a polymer material and/or transparent radio conductive polymer mixture. Preferably a polymer mixture will be used: PEDOT: PSS, a polymer of low conductivity, namely a mixture of poly (3,4-ethylenedioxythiophene (PEDOT) and sodium polystyrene sulfonate (PSS).

After a certain number of radio transparency tests and beam degradation tests passing through the sensor 3, the applicant advantageously selected a thickness of the antenna 10 comprises between 2 and 20 microns, and preferably between 5 and 10 microns.

In the photograph, the polymer is printed with a thickness of 6 microns. The substrate used is 3 mm thick glass, which has a permittivity Er=5.6 and a dielectric dissipation factor tan 6=0.02. The glass substrate is only given by way of example and the PEDOT: PSS can be deposited on flexible substrates of plastic type (PTE), substrates of conventional industrial electronics (kapton) but also on substrates also biocompatible (parylen) by means of a specific surface treatment prior to deposition of the conductive polymer.

Referring this time to FIG. 4, there is shown a sensor 3 in schematic form with its different dimensions. Advantageously, the height and the width of the antenna 10 are of the order of 30 to 60 mm. The conductivity of the PEDOT: PSS antenna is of the order 1.5 104 S/m with an antenna thickness of the order of 6 microns. The first transmission means 8 thus make it possible, given the geometry of the antenna, the conductivity and the deposit thickness of the conductive material, a reading distance of the order of one meter.

The area occupied by the antenna 10 of the sensor 3 is very large compared to the total surface of the sensor 3, some parts of which not transparent radio are. Advantageously, each sensor 3 has a radiofrequency surface at least equal to 95% of its total surface, so as not to substantially modify the action of the rays on the patient and in particular not to significantly affect the quality of the radiological image produced.

The signals transmitted by each antenna 10 comprise both the identification of the sensor 3 and therefore its position on the matrix 2 and a flux level. The flow measurement means 6 therefore allow to measure a flow level for a given sensor. The data are then transmitted to the processing means 9 by the second transmission means 8. These second transmission means 8 may be made by well-known transmission means and in particular by wired or remote radio means. In another embodiment, the first transmission means 7 send the data directly to a set consisting of a flowmeter (or equivalent) and processing means 9.

The processing means 9 receive the flow measurement for each sensor 3, from these data, the processing means 9 calculate the cumulative doses received by predetermined areas of the matrix during the operation. These areas can constitute in the detection zone of a sensor 3 or of a set of sensors 3.

Advantageously, the processing means 9 comprise display means 12 in the form of a map of the dose accumulation levels by zones of the matrix 2. The real-time display allows the practitioner to react very quickly depending on the data displayed, possibly to stop the beam or adjust the power or direction of the latter.

In an advantageous embodiment, the processing means 9 further comprise means for recording the flow measurements in order to cumulate the doses on at least two radiology operations.

In another embodiment, the matrix comprises several layers of sensors 3, superimposed or not, and the processing means 9 allow to calculate the doses not on the surface but on the volume, by reconstituting the volume from the different layers of sensor 3.

The measuring device 1 as described above thus constitutes a powerful solution for measuring the doses received by a patient allowing the practitioner to precisely adjust the doses delivered to the patient while preserving the latter from the risks of overexposure.

Of course, other features of the invention could also have been envisaged without departing from the scope of the invention defined by the claims below.

By way of example, in an advantageous embodiment, the processing means comprise means of warning of exceeding at least one radiation threshold per predetermined area of the matrix.

1. A device for measuring a radioelectric dose on a surface, in particular for measuring the radiological dose of a patient during a radiological operation, comprising:
a matrix with sensors equipped with transparent radio antennas able to create an electrical signal corresponding to the electromagnetic radiation received,
measurement means for the electrical fluxes emitted by each sensor, and
processing means of flux measurements for calculate the accumulation of doses received by predetermined areas of the matrix.

2. The device for measuring a radioelectric dose on a surface according to claim 1, wherein the processing means comprise means for displaying in the form of a map the dose accumulation levels by areas of the matrix.

3. The device for measuring a radioelectric dose on a surface according to claim 1, in which wherein the processing means comprise means for recording the flow measurements in order to cumulate the doses on at least two radiology operations.

4. The device for measuring a radioelectric dose on a surface according to claim 1, wherein the processing means comprise means for warning the exceeding of at least one radiation threshold per predetermined area of the matrix.

5. The device for measuring a radioelectric dose on a surface according to claim 1, wherein the matrix is integrated with a flexible transparent radiating mat adapted to conform to the shapes of a patient.

6. The device for measuring a radioelectric dose on a surface according to claim 5, wherein the mat comprises locating means adapted to allow positioning on the patient.

7. The device for measuring a radioelectric dose on a surface according to claim 1, wherein the radio sensor and the flux measuring means comprise the radio-transparent antenna associated with an RFID type transmitter.

8. The device for measuring a radioelectric dose on a surface according to claim 7, wherein the antenna is made of a polymer material and/or transparent radio conductive polymer mixture.

9. The device for measuring a radioelectric dose on a surface according to claim 7, in which the material of the antenna is a mixture of poly 3,4-ethylenedioxythiophene (PEDOT) and sodium polystyrene sulfonate (PSS).

10. The device for measuring a radioelectric dose on a surface according to claim 7, wherein the thickness of the antenna is between 2 and 20 microns, and preferably between 5 and 10 microns.

11. The device for measuring a radioelectric dose on a surface according to claim 1, wherein each sensor has a radiofrequency area at least equal to 95% of its total area.
